(19) United States
(12) Patent Application Publication
(10) Pub. No.: US 2013/0296801 A1
(43) Pub. Date: Nov. 7, 2013

(54) SYRINGE PROVIDED WITH NEEDLE, AND METHOD FOR PRODUCING SYRINGE PROVIDED WITH NEEDLE

(71) Applicant: Kouichi AKIYAMA, Nakakoma-gun, Yamanashi (JP)

(72) Inventors: Masafumi Takemoto, Fujinomiya-shi (JP); Kouichi Akiyama, Nakakoma-gun (JP)

(73) Assignee: TERUMO KABUSHIKI KAISHA, Shibuya-ku, Tokyo (JP)

(21) Appl. No.: 13/980,863

(22) PCT Filed: Nov. 25, 2011
(86) PCT No.: PCT/JP2011/077220

§ 371 Date: Jul. 19, 2013

(30) Foreign Application Priority Data
Jan. 19, 2011 (JP) 2011008441

(51) Int. Cl. A61M 5/34

(52) U.S. Cl. CPC A61M 5/34
USPC 604/240; 29/428

(57) ABSTRACT

Embodiments of the present invention provide a syringe with a needle and method of forming thereof. The syringe with the needle includes a needle tube having a needle tip to puncture a living body and a syringe. The syringe further includes a main body filled with a drug, a fixing section for fixing the needle tube in a state where the needle tip of the needle tube being protruded, and a connecting section at which the fixing section and the main body are connected. The connecting section is provided with a holding hole into which a proximal portion of the needle tube is inserted. The needle tube and the fixing section are fixed by forming at least a portion of the fixing section from a resin composed of essentially the same material as the syringe, after the proximal portion of the needle tube has been inserted into the holding hole.

This application is the national phase entry under U.S.C. §371 of International Application No. PCT/JP2011/077220, filed on Nov. 25, 2011, which claims priority under 35 U.S.C. §119 to Japanese Patent Application No. JP2011-008441, filed on Jan. 19, 2011, the contents of which are hereby incorporated by reference in their entirety for all purposes.

### TECHNICAL FIELD

The present invention relates to a syringe provided with a needle such that a needle tube and a syringe are integrally formed by molding without using any adhesive, and to a method for producing the syringe provided with a needle.

### BACKGROUND OF THE INVENTION

In general, a syringe provided with a needle is composed of a needle tube provided at a distal portion thereof with a needle tip capable of puncturing a living body, and a needle hub which holds the needle tube with the needle tip of the needle tube being protruded. In addition, the syringe provided with a needle is configured by connecting a syringe to the needle hub. Hitherto, the needle tube has been fixed to the needle hub by use of an adhesive, in the state of being inserted and passed in an insertion hole in the needle hub. See, for example, Japanese Patent Laid-open No. 2006-116163 (hereinafter Patent Document 1).

In recent years, a syringe provided with a needle has been proposed in which a needle hub is eliminated and a needle tube is fixed directly to a syringe to be filled with a drug. In this syringe provided with a needle, also, the needle tube is fixed to the syringe by use of an adhesive, like in the case of the syringe provided with a needle according to the related art.

However, the syringe is filled with a drug in some cases. Therefore, there is a risk of contact (liquid contact) of an adhesive, used to fix the needle tube and the syringe to each other, with the drug filled in the syringe, leading to the problem that the drug is adversely affected. In recent years, therefore, there has been a need for a technology of integrally forming a needle tube and a syringe by insert molding, as a novel fixing method in which no adhesive is used.

In addition, in the method of simultaneously forming the needle tube and the syringe by insert molding, complicated parting lines of mold are involved for preventing the needle tube from being damaged. As a result, a mold with a complicated structure is required. Thus, there is a problem that the steps for producing the syringe provided with a needle become complicated.

Furthermore, a core pin for forming a tube hole of the syringe at the time of insert molding has to be provided with a portion for supporting the needle tube. After molding, therefore, the tube hole is formed therein with a space at the part where the needle tube has been supported. The space finishes playing a role when the insert molding is over. After the insert molding, therefore, the space does not fulfill any special function and is unnecessary. As a result, the space becomes a dead volume, in which the drug would be left uninjected.

In consideration of the above-mentioned problems, it is an object of the present invention to provide a syringe provided with a needle such that a needle tube and a syringe can be integrally formed by molding without using any adhesive, and a method for producing the syringe provided with a needle.

### SUMMARY OF THE INVENTION

In order to solve the above-mentioned problems and to attain the object of the present invention, a syringe provided with a needle according to the embodiments of the present invention includes a needle tube and a syringe. The needle tube has a needle tip capable of puncturing a living body. The syringe includes a main body to be filled with a drug, a fixing section which is formed to be continuous with the main body and fixes the needle tube with the needle tip of the needle tube being protruded, and a connecting section at which the fixing section and the main body are connected. The connecting section of the syringe is provided with a holding hole in which a proximal portion on the side opposite to the needle tip of the needle tube is held. The needle tube is fixed to the fixing section by forming at least a part of the fixing section from a resin composed of essentially the same material as the syringe after the proximal portion of the needle tube is inserted into the holding hole.

In addition, a method for producing a syringe provided with a needle according to the present invention includes the following steps:

forming a syringe to be filled with a drug, the syringe having a connecting section at which a fixing section for fixing a needle tube having a needle tip capable of puncturing a living body is connected;

inserting a proximal portion, on the side opposite to the needle tip, of the needle tube into a holding hole provided in the connecting section of the syringe; and

forming at least a part of the fixing section from a resin composed of essentially the same material as the syringe, thereby to fix the needle tube to the fixing section.

According to the embodiments of the present invention, the proximal portion of the needle tube is inserted into the holding hole of the syringe, thereby supporting the needle tube, and a resin is injected to form a part of the fixing section, thereby fixing the needle tube and syringe. Therefore, a mold for forming the syringe does not have to be provided with a support portion for supporting the needle tube. Accordingly, dead volume can be reduced. Furthermore, since the space between the needle tube and the fixing section is filled up with the resin, the needle tube can be fixed without using any adhesive. Besides, since the resin is substantially the same material as the syringe, good bonding between them can be obtained.

### BRIEF DESCRIPTION OF DRAWINGS

FIG. 1 is a perspective view showing a first embodiment of the syringe provided with a needle according to the present invention.

FIG. 2 is a perspective view showing an essential part of the first embodiment of the syringe provided with a needle according to the present invention.

FIG. 3 is a sectional view showing a syringe according to the first embodiment of the syringe provided with a needle of the present invention.

FIG. 4 is a sectional view showing the state wherein a needle tube is mounted to the syringe according to the first embodiment of the syringe provided with a needle of the present invention.

FIG. 5 is a sectional view showing the state wherein a resin is injected into the syringe according to the first embodiment of the syringe provided with a needle of the present invention.

FIG. 6 is a perspective view of the state wherein a cap is attached to the first embodiment of the syringe provided with a needle of the present invention.

FIG. 7 is a sectional view of the syringe provided with a needle and the cap shown in FIG. 6.

FIG. 8 is a perspective view showing the state wherein a needle tube according to a second embodiment of the syringe provided with a needle of the present invention is supported.

FIG. 9 is a perspective view showing an essential part of the second embodiment of the syringe provided with a needle of the present invention.

FIGS. 10A and 10B show a syringe according to a third embodiment of the syringe provided with a needle of the present invention, wherein FIG. 10A is a side view of the syringe, and FIG. 10B is a sectional view taken along line D-D of FIG. 10A.

FIGS. 11A and 11B show the state in which a needle tube in the third embodiment of the syringe provided with a needle of the present invention is fixed, wherein FIG. 11A is a side view of the syringe provided with a needle, and FIG. 11B is a sectional view taken along line F-F of FIG. 11A.

### DETAILED DESCRIPTION OF THE INVENTION

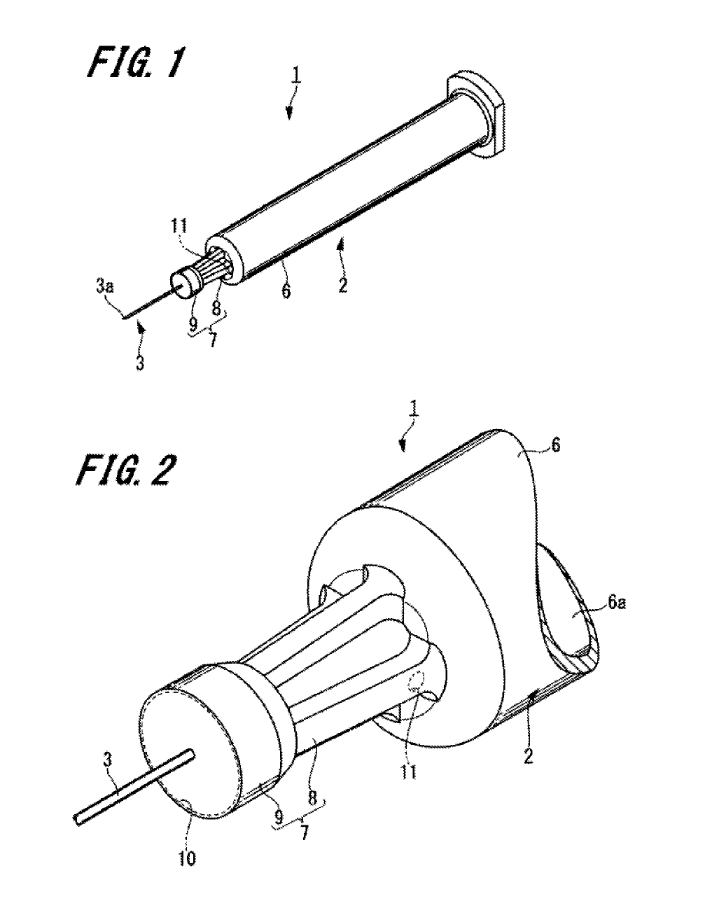

In the following, various aspects of the syringe provided with the needle according to the embodiments of the present invention will be described with reference to FIGS. 1 to 11. Incidentally, members shown in common in the drawings are denoted by the same reference signs. Besides, the present invention is not to be restricted to the following embodiments.

Incidentally, description will be made in the following order:

1. First Embodiment
1-1. Configuration Example of Syringe with Needle
1-2. Assemblage of Syringe with Needle

2. Second Embodiment

3. Third Embodiment

### 1. First Embodiment

1-1. Configuration Example of Syringe with Needle

First, a syringe provided with a needle according to a first embodiment of the present invention will be described with reference to FIGS. 1 to 7.

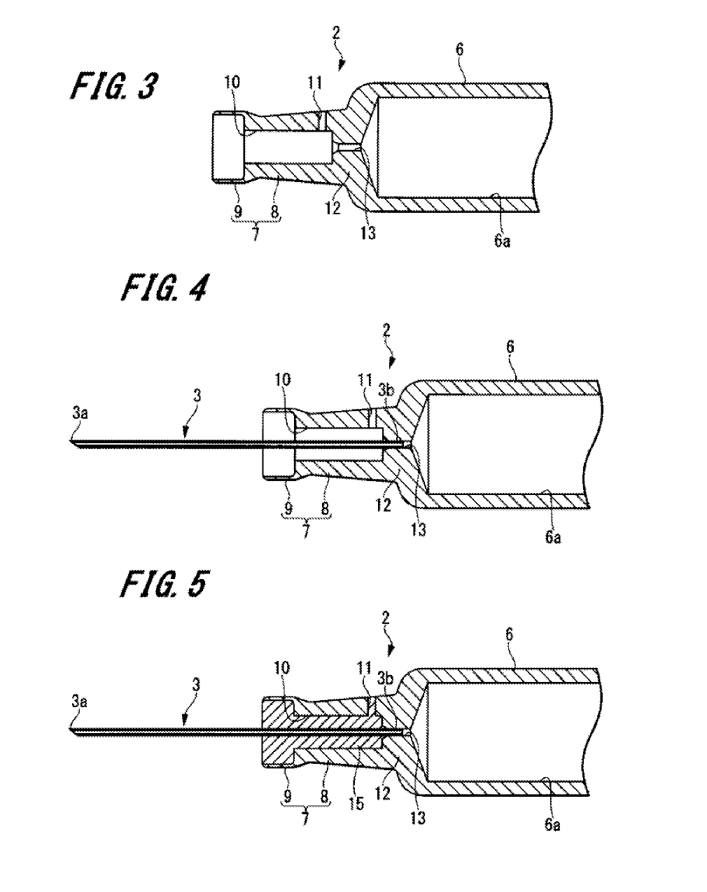

FIG. 1 is a perspective view showing a syringe provided with a needle according to the present embodiment, and FIG. 2 is a perspective view showing an essential part of the syringe provided with a needle according to the present embodiment. FIG. 3 is a sectional view showing an outer tube according to the syringe provided with a needle of the present embodiment. FIG. 4 is a sectional view showing the state in which a needle tube is mounted on the outer tube, and FIG. 5 is a sectional view showing the state in which a resin has been injected into the outer tube.

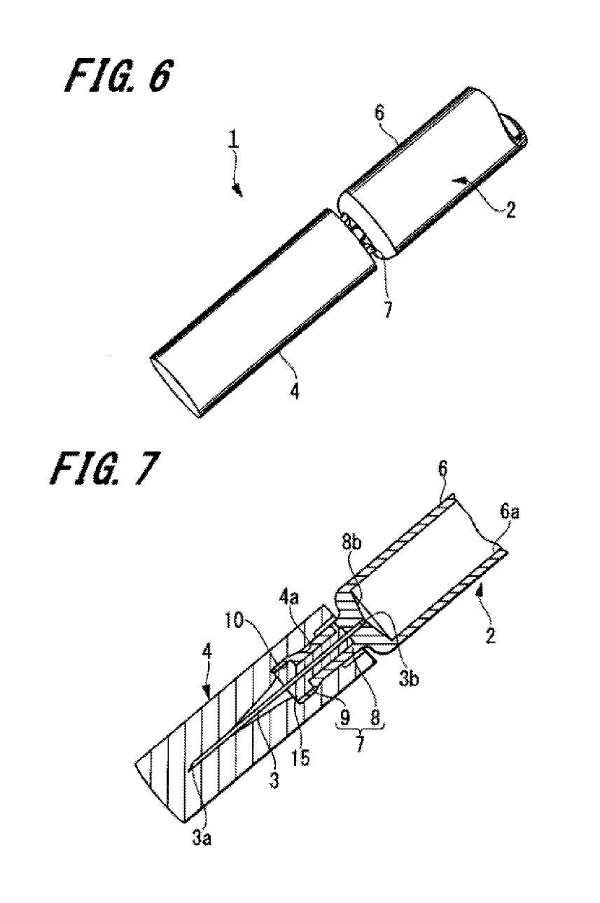

The syringe 1 provided with a needle is used to puncture a surface of a skin with a needle tip and to inject a drug into a living body. As shown in FIG. 1, the syringe 1 provided with a needle includes a syringe 2 to be filled with a drug, and a needle tube 3 fixed to the syringe 2. In addition, a cap 4 (see FIGS. 6 and 7) is mounted to the syringe 1 provided with a needle.

### Needle Tube

First, the needle tube 3 will be described. As the needle tube 3, one having a size of 10 to 33 gauge (outside diameter: 3.5 to 0.2 mm), preferably 16 to 33 gauge (outside diameter: 1.7 to 0.2 mm), according to the ISO medical needle tube standard (ISO 9626:1991/Amd. 1: 2001(E)), is used. As shown in FIG. 4A, the needle tube 3 is formed at one axial-directional end thereof with a cutting edge surface for making the needle tip 3a have an acute angle. The needle tip 3a on one side in the axial direction of the needle tube 3 is made to puncture a living body.

Examples of the material for the needle tube 3 include stainless steel, but this is not restrictive. Other metals such as aluminum, aluminum alloys, titanium, and titanium alloys can also be used. In addition, the needle tube 3 is not restricted to a straight needle but may be a tapered needle of which at least a part is in a tapered shape. It suffices that the tapered needle is greater in diameter at a distal portion thereof than at an end portion of the needle tip 3a and that an intermediate portion of the needle has a tapered structure. Besides, the sectional shape of the needle tube 3 is not restricted to a circle but may be a polygon such as a triangle.

Furthermore, a surface on the needle tip 3a side of the needle tube 3 is coated with a coating agent such as silicone resin or fluoro-resin. This ensures that when the needle tube 3 punctures a living body, the friction between the skin and the needle tube can be reduced, whereby the pain attendant on the puncture can be alleviated.

The needle tube 3 is fixed to the syringe 2, with the needle tip 3a protruding to the outside. As shown in FIG. 4, the needle tip 3a on one side in the axial direction of the needle tube 3 protrudes from a distal portion of a fixing section 7 of the syringe 2 which will be described later.

### Syringe

Now, the syringe 2 will be described below.

As shown in FIG. 3, the syringe 2 is provided with a main body 6 to be filled with a drug, and the fixing section 7 continuous with the main body 6. Between the main body 6 and the fixing section 7 is provided a connecting section 12. The main body 6 is formed in a substantially cylindrical shape. The fixing section 7 is formed continuously on one side in the axial direction of the main body 6, while the other end in the axial direction of the main body 6 is opening. In the case of a prefilled syringe, a tube hole 6a of the main body 6 is filled with a drug, and a gasket as a pusher is pluggingly inserted from the other side in the axial direction of the main body 6.

Incidentally, while in this embodiment, the main body 6 of the syringe 2 is substantially cylindrical in shape, the shape of the main body 6 in other embodiments may be a hollow tetragonal prism or hexagonal prism.

As shown in FIGS. 2 and 3, the fixing section 7 protrudes from one side in the axial direction of the main body 6 along the axial direction. The fixing section has a joining section 8 continuous with one axial-directional end of the main body 6, and a distal portion 9 continuous with the joining section 8. In addition, the fixing section 7 is provided therein with an insertion hole 10 bored in the range from the distal portion 9 to the main body 6. Into the insertion hole 10, the needle tube 3 is inserted along the axial direction thereof.

The diameter of aperture of the insertion hole 10 is set to be sufficiently greater than the diameter of the needle tube 3 so as to make it possible to cope with needle tubes 3 having different diametral sizes. In addition, the insertion hole 10 may be formed in a tapered shape such that its diameter increases along the direction from the main body toward the distal portion 9. This facilitates insertion of the needle tube 3 into the insertion hole 10, whereby an assembling operation can be simplified.

Incidentally, since the insertion hole 10 is filled up with a resin 15 described later, the shape of the insertion hole 10 can be appropriately set according to the shape of the needle tube 3.

The connecting section 12, which is continuous with the insertion hole 10 and at which the fixing section 7 and the main body 6 are connected, is formed therein with a holding hole 13. The holding hole 13 is opening in a substantially cylindrical shape on the main body 6 side, and a proximal portion 3b as the other end in the axial direction of the needle tube 3 is inserted therein (see FIG. 4). With the proximal portion 3b of the needle tube 3 inserted in the holding hole 13, the needle tube 3 is supported, and the tube hole 6a of the main body 6 and the needle tube 3 communicate with each other via the holding hole 13. Incidentally, the holding hole 13 has its portion on the fixing section 7 side opening in a tapered shape such that the diameter thereof continuously increases toward one side in the axial direction thereof. This ensures that the proximal portion 3b of the needle tube 3 can be easily inserted into the holding hole 13, and needle tubes 3 having different diametral sizes can be supported in the holding hole 13.

The joining section 8 is cross-shaped in section orthogonal to the axial direction of the main body 6. In addition, the diameter on the distal portion 9 side of the joining section 8 is smaller than the diameter of the distal portion 9. Therefore, the fixing section 7 is shaped to be constricted at the portion of connection between the joining section 8 and the distal portion 9. Incidentally, while the joining section 8 becomes smaller in diametral size along the direction of going away from the main body 6 in the present embodiment, it may be continuous and remain constant in diametral size.

Furthermore, the joining section 8 is formed with an injection hole 11 penetrating from the circumferential surface to the insertion hole 10, as one example of an injection section. The injection hole 11 is provided rather on the main body 6 side than on the distal portion 9 side, with reference to the axial-directional center of the joining section 8. In addition, as shown in FIG. 5, the resin 15 is injected into the injection hole 11 and the insertion hole 10. With the space between the needle tube 3 inserted in the insertion hole 10 and the insertion hole 10 filled with the resin 15, a remaining part of the fixing section 7 is formed, and the needle tube 3 is thereby fixed to the fixing section 7 of the syringe 2.

The resin 15 is composed of essentially the same material as the syringe 2. Therefore, good bonding to the syringe 2 can be obtained, and the needle tube 3 can be firmly fixed to the syringe 2. Besides, a mark is less liable to be left at the aperture of the insertion hole 10 or the injection hole 11, which contributes to better appearance quality of the syringe 2.

Incidentally, while an example wherein the joining section 8 is cross-shaped for ensuring a thin material thickness and securing strength has been described in the present embodiment, this is not restrictive. For instance, the shape of the joining section 8 may be roughly in the shape of a cylinder, a quadrangular prism or a hexagonal prism.

The distal portion 9 is provided on the side of one axial-directional end of the joining section 8, or on the side of the distal end of the fixing section 7. The distal portion 9 is formed in a substantially cylindrical shape. The outer circumferential surface of the distal portion 9 comes into close contact with the inner circumferential surface of the cap 4, which will be described later.

Incidentally, while an example wherein the distal portion 9 is formed in a substantially cylindrical shape has been described above, the shape of the distal portion 9 may also be a quadrangular prism or a hexagonal prism insofar as the shape corresponds to the shape of a tube hole 4a in the cap 4.

As the material of the syringe 2 configured as above, the following various resins can be mentioned. Examples of the resins include polyvinyl chloride, polyethylene, polypropylene, cyclic polyolefins, polystyrene, poly(4-methylpentene-1), polycarbonate, acrylic resin, acrylonitrile-butadiene-styrene copolymer, polyesters such as polyethylene terephthalate, etc., butadiene-styrene copolymer, polyamides (e.g., nylon 6, nylon 6,6, nylon 6,10, nylon 12). Among these, such resins as polypropylene, cyclic polyolefins, polyesters, and poly(4-methylpentene-1) are preferably used. Incidentally, the material of the syringe 2 is preferably transparent, for securing inside visibility.

The drug to be filled into the syringe 2 may be any of those drugs which are commonly used as injections. Examples of the drug include protein drugs such as antibodies, etc., peptide drugs such as hormones, etc., nucleic acid drugs, cell drugs, blood preparations, vaccines for prevention of various infectious diseases, carcinostatic agents, anesthetics, narcotic drugs, antibiotics, steroid preparations, proteinase inhibitors, heparin, saccharide injections such as glucose, etc., electrolyte correction injections such as sodium chloride, potassium lactate, etc., vitamin preparations, lipid emulsions, contrast media, and stimulants.

### Cap

The cap 4 will be described below with reference to FIGS. 6 and 7.

FIG. 6 is a perspective view showing the state wherein the cap 4 is attached to the syringe 1 provided with a needle, and FIG. 6 is a sectional view showing the syringe 1 provided with a needle and the cap 4 shown in FIG. 7.

As shown in FIGS. 6 and 7, the cap 4 is formed in a substantially cylindrical shape, with its one axial-directional end opening and with its other axial-directional end closed. The cap 4 is formed, for example, from an elastic material such as rubber or elastomer. In addition, as shown in FIG. 6, the cap 4 is attached to one side in the axial direction of the syringe 2 in such a manner as to cover the needle tip 3a of the needle tube 3 and the fixing section 7 of the syringe 2. Besides, as shown in FIG. 7, the needle tip 3a side of the needle tube 3 and the fixing section 7 are inserted in the tube hole 4a of the cap 4.

Incidentally, the inside diameter of the tube hole 4a of the cap 4 is set to be approximately equal to or slightly smaller than the outside diameter of the distal portion 9 of the fixing section 7. Therefore, when the cap 4 is mounted to the syringe 2, the outer circumferential surface of the distal portion 9 of the fixing section 7 makes close contact with the inner circumferential surface of the cap 4. This ensures that the needle tip 3a side of the needle tube 3, on the distal side relative to the distal portion 9 of the fixing section 7, is hermetically sealed by the distal portion 9 and the inner circumferential surface of the cap 4. As a result, adhesion of bacteria to the needle tip 3a is prevented.

In addition, the inner circumferential surface of the cap 4 fastens, by its elastic force, the portion of connection between the distal portion 9 and the joining section 8 of the fixing section 7, namely, the constricted portion. Consequently, the inner circumferential surface of the cap 4 and the constricted portion of the fixing section 7 are engaged with each other, whereby the cap 4 is prevented from being disengaged from the syringe 2 during transportation.

1-2. Assemblage of Syringe with Needle

Referring next to FIGS. 3 to 5, a method of assembling the syringe 1 provided with the needle according to the embodiments of the present invention will be described.

First, as shown in FIG. 3, the syringe 2 is molded by use of a mold. Next, as shown in FIG. 4, the needle tube 3 is inserted into the insertion hole 10 of the fixing section 7 of the syringe 2, and the proximal portion 3b of the needle tube 3 is inserted into the holding hole 13. In this instance, since the aperture on the distal portion 9 side of the holding hole 13 is tapered, the proximal portion 3b of the needle tube 3 can be easily inserted into the holding hole 13, which permits easy assemblage of the syringe 1 provided with a needle. As a result, the needle tube 3 is held on the syringe 2.

Incidentally, the proximal portion 3b of the needle tube 3 may be chamfered. This promises easier insertion of the needle tube 3 into the holding hole 13. In addition, with the main body 6 molded separately from the needle tube 3 and with the needle tube 3 supported by the holding hole 13, a core pin for forming the tube hole 6a at the time of molding the main body 6 does not have to be provided with a portion for supporting the needle tube 3. As a result, as compared with a conventional syringe provided with a needle such that a needle tube and a syringe are formed at a time, dead volume of the tube hole 6a can be reduced and, therefore, the amount of the liquid drug left uninjected can be reduced.

Next, the resin 15 composed of essentially the same material as the syringe 2 is injected into the space between the needle tube 3 and the insertion hole 10 through the injection hole 11 provided in the fixing section 7, thereby forming a part of the fixing section 7. Since the syringe 2 and the resin 15 are formed from essentially the same material, good bonding between them can be obtained. When the resin 15 is solidified, the remaining part of the fixing section 7 is formed, whereby the needle tube 3 can be firmly fixed to the syringe 2, and the assemblage of the syringe 1 provided with a needle is completed.

Incidentally, at the time of injecting the resin 15, the operation is preferably carried out with the syringe 2 set in the mold. Here, the operation of injecting the resin 15 is conducted with a high pressure being applied to the resin 15, for filling up the insertion hole 10 with the resin 15 without leaving any space. This means that a pressure generated at the time of injecting the resin 15 is exerted on the syringe 2. With the injecting operation carried out with the syringe 2 set in the mold, therefore, the syringe 2 can be prevented from being deformed by the pressure generated at the time of injection of the resin 15. In addition, since the resin can be filled into the insertion hole 10 of the syringe 2 in the proper quantity, the appearance quality of the syringe 1 provided with a needle can be enhanced.

The injection hole 11 is formed at a position rather on the main body 6 side than on the distal portion 9 side with reference to the axial-directional center of the joining section 8. Therefore, the resin 15 can be securely supplied to the side of the proximal portion 3b of the needle tube 3, so that a base portion of the needle tube 3 can be firmly fixed to the syringe 2. Furthermore, with the resin 15 filled also into the injection hole 11, no mark of the injection hole 11 would be left on a side surface of the joining section 8. This promises enhanced appearance quality of the syringe 1 provided with a needle.

Besides, since the needle tube 3 is fixed to the syringe 2 by a method wherein the resin 15 composed of substantially the same material as the syringe 2 is injected to thereby form a part of the fixing section 7, the syringe 1 provided with a needle can be assembled without using any adhesive. Accordingly, an adhering step to be conducted by use of an adhesive can be omitted. In addition, since no adhesive is used, there is no fear that an adhesive might come into contact with the drug filled in the syringe 2, thereby adversely affecting the drug.

### 2. Second Embodiment

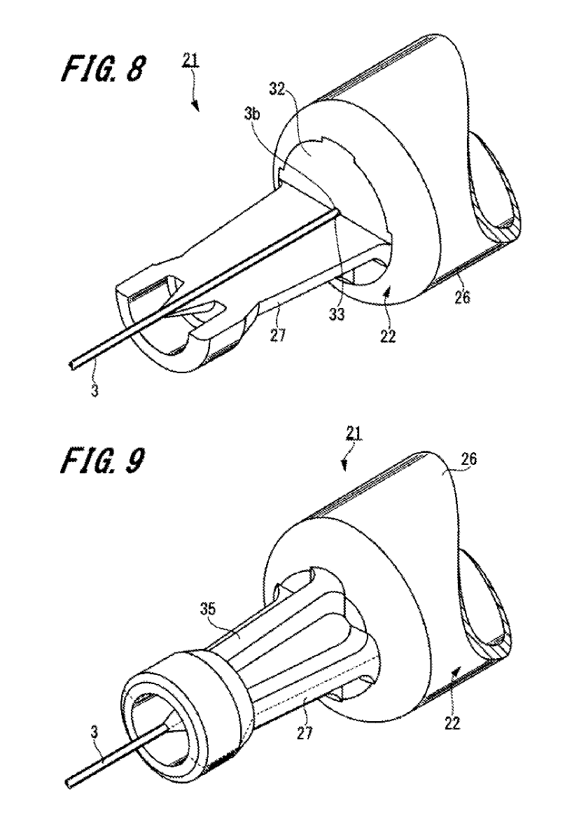

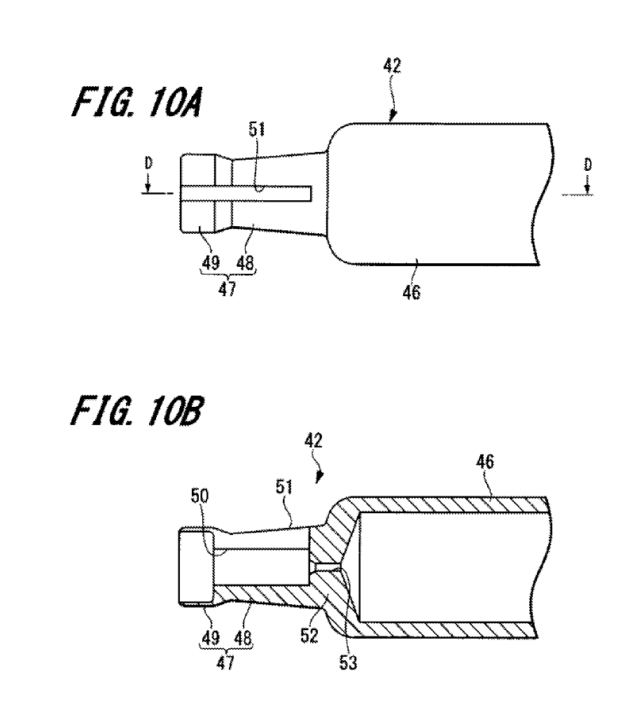

In the following, a second embodiment of the syringe, provided with a needle according to the embodiments of the present invention, will be described with reference to FIGS. 8 and 9.

FIGS. 8 and 9 are perspective views showing an essential part of a syringe provided with a needle according to the second embodiment.

The syringe 21 provided with the needle according to the second embodiment differs from the first embodiment in that a configuration is used for fixing a fixing section and the needle tube. Here, therefore, the fixing section will be described; on the other hand, the same parts as those of the syringe 1 provided with a needle are denoted by the same reference symbols as used above, and overlapping descriptions of them will be omitted.

As shown in FIG. 8, a syringe 22 has a main body 26 to be filled with a drug, and a fixing section 27 continuous with the main body 26. Between the main body and the fixing section 27 is provided a connecting section 32. The fixing section 27, before fixation of a needle tube 3, has a loss of about one half thereof along the axial direction.

The needle tube 3 is supported on the syringe 22 by inserting a proximal portion 3b thereof in a holding hole 33 provided in the connecting section 32. In addition, as shown in FIG. 9, the loss portion of the fixing section is formed by use of a resin 35 composed of substantially the same material as the syringe 22, whereby the needle tube 3 is fixed to the syringe 22.

Incidentally, while an example wherein the fixing section 27 has a loss of about one half thereof has been described in the second embodiment, this is not restrictive, and the fixing section 27 may have a loss of one third thereof along the axial direction. Or, alternatively, a configuration may be adopted wherein the fixing section 27 is totally absent, and the fixing section 27 as a whole is molded by use of a resin comprised of essentially the same material as the syringe 22, after insertion of the proximal portion 3b of the needle tube 3 into the holding hole 33. In other words, it suffices that the proximal portion 3b of the needle tube 3 can be inserted into the holding hole 33 in the connecting section 32 in the condition where the fixing section 27 is absent.

The other configurations are the same as those of the syringe 1 provided with a needle according to the first embodiment described above, and, therefore, descriptions of them are omitted. According to the syringe 21 provided with a needle configured in this way, also, the same operation and effect as those of the syringe 1 provided with a needle according to the first embodiment described above can be obtained.

### 3. Third Embodiment

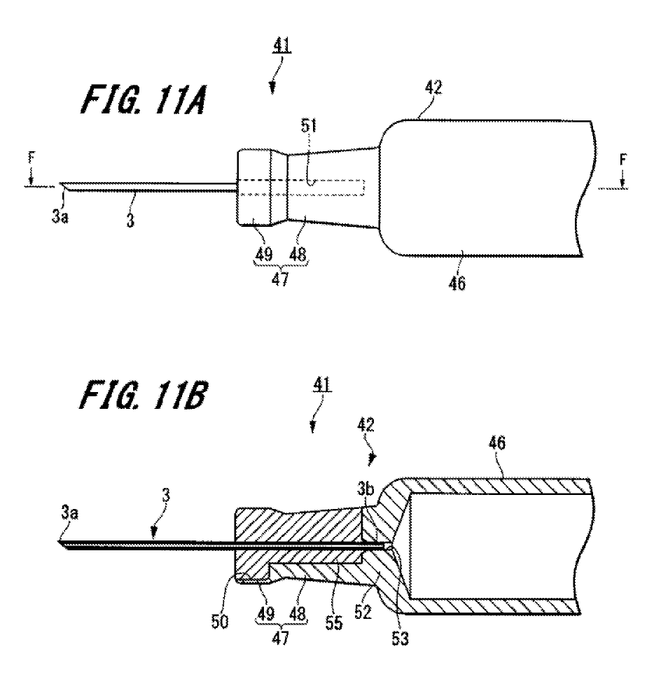

Now, a third embodiment of the syringe provided with a needle according to the present invention will be described below, with reference to FIGS. 10 and 11.

FIGS. 10 and 11 are illustrations of an essential part of a syringe provided with a needle according to the third embodiment.

The syringe 41 provided with a needle according to this third embodiment differs from the syringe 1 provided with a needle according to the first embodiment in that an injection section is configured as a groove. Here, therefore, a fixing section will be described; on the other hand, the same parts as those of the syringe 1 provided with a needle are denoted by the same reference signs as used above, and overlapping descriptions of them will be omitted.

As shown in FIG. 10A, a fixing section 47 of a syringe 42 has a joining section 48 continuous with one axial-directional end of a main body 46, and a distal portion 49 continuous with the joining section 48. In addition, as shown in FIG. 10B, the fixing section 47 is provided with an insertion hole 50 opening in the region ranging from the distal portion 49 to the main body 46. Besides, a needle tub 3 is inserted into the insertion hole 50 along the axial direction thereof.

The joining section 48 and the distal portion 49 are provided with an injection groove 51 representing another example of the injection section. The injection groove 51 is a cutout which penetrates from the circumferential surfaces of the joining section 48 and the distal portion 49 to the insertion hole 50 and which is formed to be continuous from the joining section 48 to the distal end of the distal portion 49. A resin 55 is injected into the space between the needle tube 3 and the insertion hole 50 via the injection groove 51, in the condition where the proximal portion 3b of the needle tube 3 is inserted in a holding hole 53 in a connecting section 52, as shown in FIGS. 11A and 11B. As a result, a part of the fixing section 47 is formed, whereby the needle tube 3 can be firmly fixed to the syringe 42.

The other configurations are the same as those of the syringe 1 provided with a needle according to the first embodiment described above, and, therefore, descriptions of them are omitted. According to the syringe 41 provided with a needle configured in this manner, also, the same operation and effect as those of the syringe 1 provided with a needle according to the first embodiment described above can be obtained.

Incidentally, the present invention is not to be restricted to the embodiments described above and illustrated in the drawings, and various modifications are possible within the gist of the invention as described in the claims.

1. A syringe provided with a needle, comprising:
a needle tube having a needle tip capable of puncturing a living body; and
a syringe including a main body configured to be filled with a drug, a fixing section formed continuously with the main body and fixing the needle tube such that the needle tip of the needle tube protrudes therefrom, and a connecting section at which the fixing section and the main body are connected,
wherein the connecting section of the syringe is provided with a holding hole into which a proximal part of the needle tube is inserted, the proximal part being located on an opposite side to the needle tip, and
the needle tube is fixed to the fixing section by forming at least a portion of the fixing section from a resin composed of essentially the same material as the syringe after the proximal portion of the needle tube is inserted into the holding hole.

2. The syringe provided with a needle according to claim 1, wherein the fixing section of the syringe is provided with:
an insertion hole which is continuous with the holding hole and into which the needle tube is inserted; and
an injection section for injecting the resin into a space between the insertion hole and the needle tube.

3. The syringe provided with a needle according to claim 1,
wherein the holding hole is opened in a tapered shape such that a diameter thereof increases along a direction from the main body toward the fixing section.

4. The syringe provided with a needle according to claim 2,
wherein the insertion hole is opened in a tapered shape such that a diameter thereof decreases along a direction from a distal portion of the fixing section toward the main body, in which the needle tube is protruded from the distal portion.

5. A method for producing a syringe provided with a needle, comprising steps of:
forming a syringe configured to be filled with a drug, the syringe having a connecting section at which a fixing section for fixing a needle tube having a needle tip capable of puncturing a living body is connected;
inserting a proximal portion of the needle tube into a holding hole provided in the connecting section of the syringe, the proximal portion being located on an opposite side to the needle tip; and
forming at least a part of the fixing section from a resin composed of essentially the same material as the syringe, thereby fixing the needle tube to the fixing section.

6. The method for producing a syringe provided with a needle according to claim 5,
wherein, in the step of forming the syringe, the fixing section having an insertion hole which is continuous with the holding hole and into which the needle tube is inserted and an injection section for injecting the resin is preliminarily and integrally molded with the syringe, and
in the step of fixing the needle tube to the fixing section, a part of the fixing section is molded by injecting the resin into a space between the insertion hole and the needle tube through the injection section.
